(19) United States
(12) Patent Application Publication
(10) Pub. No.: US 2023/0148872 A1
(43) Pub. Date: May 18, 2023

(54) Temperature Monitoring for Vessel Detection

(71) Applicant: Bard Access Systems, Inc., Salt Lake City, UT (US)

(72) Inventors: Steffan Sowards, Salt Lake City, UT (US); William Robert McLaughlin, Bountiful, UT (US); Anthony K. Misener, Bountiful, UT (US)

(21) Appl. No.: 17/987,698

(22) Filed: Nov. 15, 2022

Related U.S. Application Data
(60) Provisional application No. 63/280,043, filed on Nov. 16, 2021.

(51) Int. Cl. A61B 5/01; A61B 8/00; A61B 8/08; A61B 5/00

(52) U.S. Cl. CPC A61B 5/015; A61B 8/4416; A61B 8/5261; A61B 8/085; A61B 8/463; A61B 5/489; A61B 2090/365

(57) ABSTRACT

A medical system that includes a temperature scanning device configured to identify and locate blood vessels by obtaining a thermal image from a skin surface where blood flowing within blood vessels beneath the skin surface has been altered to define temperature variations of the skin. The system includes a console configured to communicate with the temperature scanning device, the console including processors and logic that, when executed by the processors, causes operations including defining the thermal image. The system may further include a camera, and/or an ultrasound probe. The thermal image may be portrayed on various forms of a display include augmented reality glasses. The thermal image may be overlayed onto a camera image and/or an ultrasound image.

### PRIORITY

This application claims the benefit of priority to U.S. Patent Application No. 63/280,043, filed Nov. 16, 2021, which is incorporated by reference in its entirety into this application.

### BACKGROUND

Intravascular medical procedures are very common. Most every patient in a hospital experiences some intravascular procedure ranging from simple intravenous drug delivery to stent installation. As such, accessing the vasculature of the patient is a routine procedure performed by various care providers, primarily nurses. Cannulating the proper blood vessel for a defined procedure may be straight forward in some instances and complicated in others. Neonatal patients at one end of the patient spectrum and geriatric patients at the other have blood vessels that in some instances are difficult to find, identify, and cannulize. Accordingly, patient care facilities and patients could benefit by lowering error rate associated with cannulating the proper blood vessels.

Medical technologies, such as ultrasound imaging, cannula tracking, and the like have helped reduce the error rate. However, these technologies require setup time and additional equipment that cost.

Disclosed herein are systems and methods that utilize temperature scanning to identify and locate blood vessels and determine other vascular conditions.

### SUMMARY

Disclosed herein is a medical system in accordance with some embodiments. The medical system generally includes a temperature scanning device configured to provide temperature data based on temperature variations of a skin surface extending across a subcutaneous target area of a patient body. The system further includes a console configured to communicate with the temperature scanning device, where the console includes one or more processors and a non-transitory computer-readable medium having stored thereon logic that, when executed by the one or more processors, causes system operations. The operations include obtaining temperature data across the target area, the target area including a subcutaneous blood vessel, wherein a cause of the temperature variations across the target area include a temperature difference of a blood temperature within the blood vessel with respect to a body tissue temperature adjacent the blood vessel.

In some embodiments, the operations further include providing an indication to the clinician based on the temperature data. In further embodiments, the operations include converting the temperature data into thermal image data, and providing a thermal image to the clinician based on the thermal image data. The thermal image may include a mapping of the blood vessels within the target area. Providing the thermal image may include includes providing a live thermal image and/or thermal image snapshots.

In some embodiments, the system further includes a temperature source configured to alter the blood temperature within the blood vessel. In some embodiments, the blood temperature is altered by placing the temperature source in contact with a target contact area of the patient, where the target contact area is located upstream with respect to a blood flow within the blood vessel. In some embodiments, the temperature source may be one of a hot pack or a cold pack, and in other embodiments, the temperature source may be a thermal contact pad of a targeted temperature management system. In some embodiments, the temperature source is an infusate delivered to the blood vessel.

The temperature variations may be the result of a temperature gradient extending along the blood vessel. In other embodiments, the temperature variations may be caused by a temperature of blood outside of the blood vessel with respect to the body tissue temperature adjacent the blood vessel. In some embodiments, the blood temperature is lower than the temperature of the body tissue. In some embodiments, the temperature variations may be caused by an infusate temperature outside of the blood vessel with respect to the body tissue temperature adjacent the blood vessel.

In some embodiments, the console is included within a housing of the temperature scanning device. In further embodiments, the display of the system is included within the housing of the temperature scanning device.

In some embodiments, the system includes a camera for providing an image of the target area and the camera may be included within the housing of the temperature scanning device. The operations may further include obtaining a camera image of the target area, and providing the thermal image superimposed atop the camera image.

In some embodiments, the system may include an ultrasound probe coupled with the console, the ultrasound probe configured to obtain an ultrasound image of the target area and the operations may further include obtaining an ultrasound image of the target area, and providing the thermal image superimposed atop the ultrasound image. In some embodiments, the temperature source is integrated into the ultrasound probe.

In some embodiments, the temperature scanning device is a hand-held device. In some embodiments, the system includes an augmented reality device, where the temperature scanning device is coupled with the augmented reality device, and an augmented reality image of the target area includes the mapping of the blood vessels overlayed onto a view of the target area.

Also disclosed herein is a method of determining a vascular condition of a patient. According to some embodiments, the method includes causing a temperature difference within a subcutaneous target area of the patient, obtaining a thermal image of a skin surface adjacent the subcutaneous target area, and determining the vascular condition based on the thermal image.

In some embodiments of the method, causing a temperature difference includes placing a temperature source in contact with the patient to alter the temperature of blood flowing through one or more blood vessels within the target area and determining the vascular condition includes identifying each of one or more blood vessels as a vein or as artery based on the location of the temperature source with respect to the target area.

In some embodiments of the method, determining the vascular condition includes determining a blood flow direction within at least one of the one or more blood vessels, and in further embodiments, determining the vascular condition includes determining a location of at least one of one or more blood vessels within the target area.

In some embodiments of the method, causing a temperature difference includes placing a temperature source in contact with the patient upstream with respect to veinous blood flow within the target area and determining the vascular condition includes identifying at least one vein within the target area.

In some embodiments of the method, causing a temperature difference includes placing a temperature source in contact with the patient upstream with respect to blood flow within one or more blood vessels passing through the target area and determining the vascular condition includes determining an extravasation within the target area, the extravasation emanating from any of the one or more blood vessels.

In some embodiments of the method, causing a temperature difference includes delivering an infusate to the patient within the target area and determining the vascular condition includes determining an infiltration of the infusate within the target area.

In some embodiments of the method, causing a temperature difference includes applying a thermal contact pad of a targeted temperature management system to the patient. In further embodiments, causing a temperature difference includes lowering the temperature of a portion of the subcutaneous target area.

These and other features of the concepts provided herein will become more apparent to those of skill in the art in view of the accompanying drawings and following description, which describe particular embodiments of such concepts in greater detail.

### DRAWINGS

FIG. 1A illustrates a medical system including temperature scanning in use with a patient in accordance with some embodiments.

FIG. 1B is an exemplary thermal image depicting an extravasation instance in accordance with some embodiments.

FIG. 2 illustrates a block diagram of a console of the medical system of FIG. 1A in accordance with some embodiments.

FIG. 3A illustrates a temperature source applied to a hand of a patient in accordance with some embodiments.

FIG. 3B illustrates a temperature source in the form of a thermal contact pad coupled with a targeted temperature management system in accordance with some embodiments.

FIG. 4A illustrates an implementation of the system of FIG. 1A including the delivery of an infusate in accordance with some embodiments.

FIG. 4B illustrates a thermal image showing the infusate of FIG. 4A flowing within a blood vessel in accordance with some embodiments.

FIG. 4C illustrates a thermal image showing the infusate of FIG. 4A infiltrating body tissue in accordance with some embodiments.

FIG. 5 illustrates a second embodiment of the medical system including an ultrasound probe in accordance with some embodiments.

FIG. 6 illustrates a third embodiment of the medical system including an augmented reality device in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

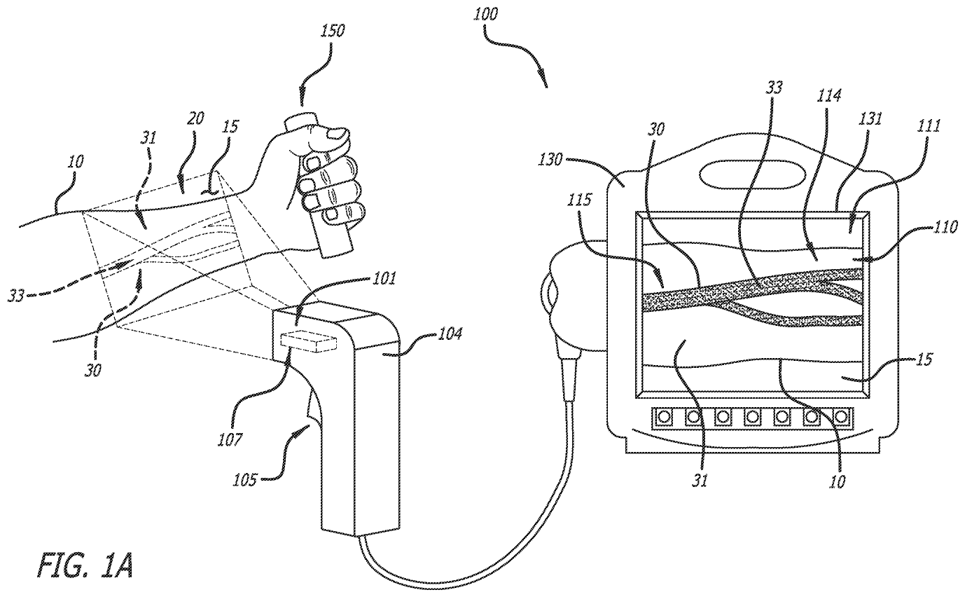

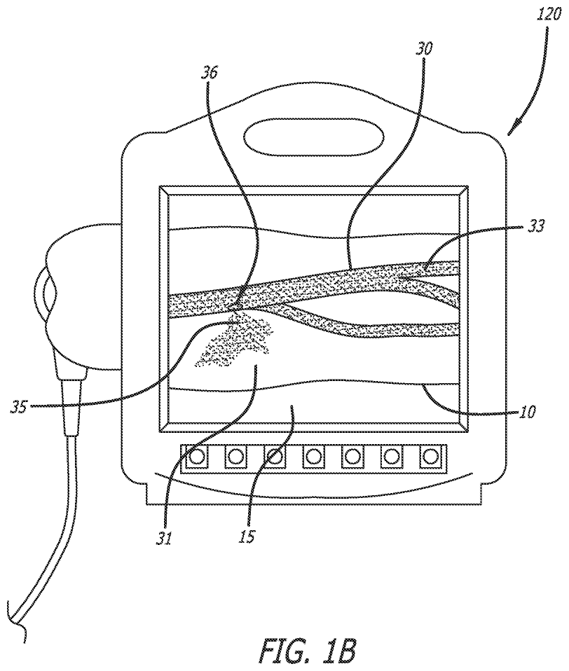

FIG. 1A illustrates a medical system configured to detect and identify blood subcutaneous blood vessels within a patient body. The medical system 100 generally includes a temperature scanning device 101 operatively coupled with a console 130, in accordance with some embodiments. The temperature scanning device 101 is configured for obtaining temperature data across a skin surface of a target area 20 of a patient 10. The temperature scanning device 101 includes a housing 104. In some embodiments, the housing 104 may facilitate grasping and holding by the hand of the clinician during use of the system 100. In some embodiments, the console 130 may be enclosed within the housing 104 of the temperature scanning device 101.

The console 130 is configured to receive the temperature data from the temperature scanning device 101 and process the temperature data in reference to blood vessels within the target area 20 as further described below. In some embodiments, the console 130 may process the temperature data to provide an indication to a medical practitioner (e.g., a clinician) regarding the location of one or more blood vessels within the target area 20. In further embodiments, the indication may include a thermal image 110 illustrating the locations. I still further embodiments, the thermal image 110 may illustrate a mapping of the blood vessels with the target area 20. In some embodiments, the console 130 includes a display 131 for portraying the thermal image 110, and in further embodiments, the display 104 may include a graphical user interface (GUI) configured for receiving input from the clinician.

The system 100 may include or be utilized in conjunction with a temperature source 150. The temperature source 150 is configured to generate a subcutaneous temperature difference within the target area 20 of the patient 10, where the subcutaneous temperature difference causes the temperature differences/variations across the skin surface. The temperature source 150 may take several forms as further described below.

According to one implementation of the system 100, the temperature source 150 causes a lowering of a blood temperature within one or more blood vessels 30. By way of one example, as illustrated in FIG. 1A, the temperature source 150 is held by the patient's hand causing a lowering of the blood temperature within the capillaries of the hand. The blood, having the lowered temperature, flows via blood vessels 30 (e.g., veins) through the target area 20. The lowered blood temperature within the blood vessels 30 results in a temperature difference between the blood vessels 30 and the surrounding body tissue 31 which is translated (i.e., migrates) to the skin surface for detection by the temperature scanning device 101. In some embodiments, the resulting temperature data is converted into the thermal image 110 that may be portrayed on the display 131. Note that the term “thermal image” as used herein includes any visual indication based on temperature data (e.g., a visual indication of the blood vessels based on the temperature data). Note that, in the illustrated implementation, the blood flowing within adjacent arterial blood vessels (not shown) is not affected by the temperature source 150 and therefore, the temperature of the arterial blood vessels is essentially the same as the temperature of the surround tissue 31. Hence, the arterial blood vessels are not detected by the temperature scanning device 101 and thus, are not portrayed in the thermal image 110.

In some implementations, the target area 20 may extend beyond the patient 10. As such, the thermal image 110 may include an environment 15 extending beyond the patient 10 where the environment 15 has a temperature that differs from the temperature of skin surface of the patient 10. Therefore, the thermal image 110, when portrayed, may include the transition between the patient 10 and the environment 15.

The thermal image 110 may help facilitate the clinician in performing a medical procedure, such as an intravascular procedure, for example. More specially, the thermal image 110 may help the clinician to identify a target blood vessel for cannulation thereby reducing the propensity of making an error when cannulizing the patient 10. The medical system 100 is useful for imaging a target blood vessel prior to a percutaneous puncture with a needle and for inserting the needle or another medical device into the target blood vessel. Indeed, the medical system 100 is shown in FIG. 1A in a general relationship to the patient P during a medical procedure. It should be appreciated that the system 100 can be useful in a variety of medical procedures other than cannulation.

It is to be noted that, while the temperature source 150 provides a cooling effect in the illustrated example, the temperature source 150 is not limited to providing a cooling effect. As such, this disclosure is broad enough to include a temperature source 150 having a temperature greater than the patient 10, the surrounding tissue 31, and/or the blood 33 flowing within the blood vessels 30.

In some embodiments, the thermal image 110 may portray a temperature gradient extending along the blood vessel 30. In other words, the thermal image 110 may depict a first temperature difference 114 and a second temperature difference 115 located downstream of the first temperature difference 114. In some instances, the first temperature difference 114 may be greater than the second temperature difference 115.

In some embodiments, although not required, the system 100 may include a camera 107. The camera 107 may be included within the housing 104 of the temperature scanning device 101 or the camera 107 may be physically separate the temperature scanning device 101. The camera 107 is communicatively coupled with the console 130 via a wired or wireless connection. The camera 107 may be configured to obtain a camera image 111 of the target area 20. In some embodiments, the camera image 111 may be portrayed in combination with the thermal image 110. In further embodiments, the camera image 111 may be superimposed atop the thermal image 110 or vice versa.

The temperature scanning device 101 may include one or more input devices 105 (e.g., control buttons) for defining operating modes of the temperature scanning device 101 or the system as a whole. In some embodiments, the input devices 105 may include a trigger for obtaining snapshots of the thermal image 110 and/or the camera image 111.

FIG. 1B illustrates another exemplary implementation of the system 100. More specifically, FIG. 1B is a thermal image 120 of an extravasation condition of the patient, i.e., a condition where blood 33 is leaking from the blood vessel 30. In such an instance, the blood 33, as cooled by the temperature source 150, is leaking into the surrounding tissue 31 through a hole 36 in the blood vessel wall causing an accumulation 35 of the blood 33 within the surrounding tissue 31. The accumulation 35 of the blood 33 causes a temperature difference between the accumulation 35 of the blood 33 and the surrounding tissue 31 that translates to the skin surface to be detected by the temperature scanning device 101.

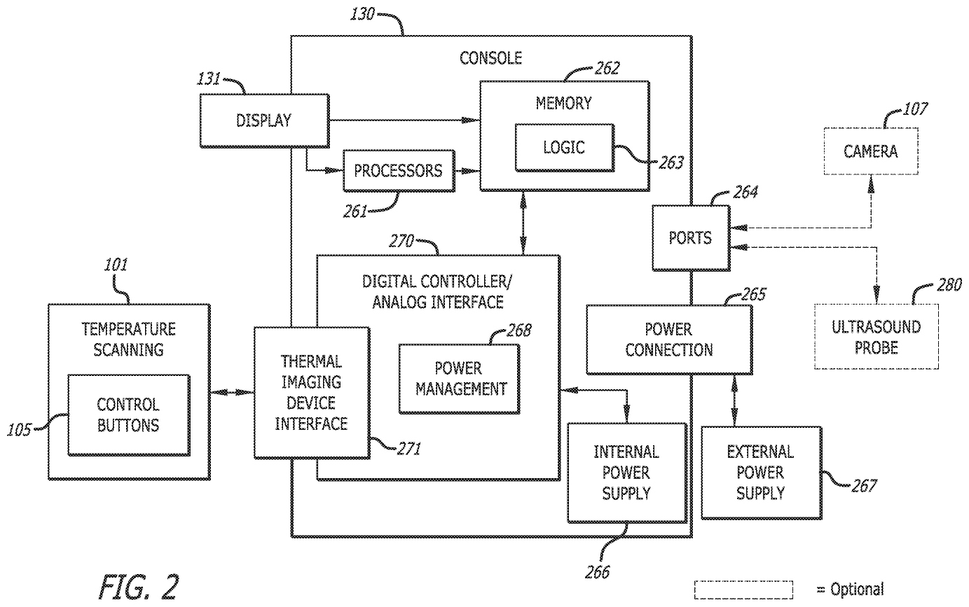

FIG. 2 illustrates a block diagram of the medical system 100 in accordance with some embodiments. As shown, the medical system 100 includes a console 130, the display 131, and the temperature scanning device 101. The console 130 includes a variety of components of the medical system 100, and it is appreciated that the console 130 can take any of a variety of forms. One or more processors 261 and memory 262 such as random-access memory (“RAM”) or non-volatile memory (e.g., electrically erasable programmable read-only memory [“EEPROM”]) is included in the console 130 for controlling functions of the medical system 100, as well as executing various logic operations or algorithms during operation of the medical system 100 in accordance with executable logic 263 therefor stored in the memory 262 for execution by the processors 261.

The medical system 100 further includes ports 124 for connection with additional components. Although not shown, additional optional components may include a printer, storage media, keyboard, etc. The ports 264 can be universal serial bus (“USB”) ports, though other types of ports can be used for this connection or any other connections shown or described herein. A power connection 265 is included with the console 130 to enable operable connection to an external power supply 267. An internal power supply 266 (e.g., a battery) can also be employed either with or exclusive of the external power supply 267. Power management circuitry 268 is included with the digital controller/analog interface 270 of the console 130 to regulate power use and distribution.

A temperature scanning device interface 271 and control buttons 105 (see FIG. 1A) included on the temperature scanning device 101 can be used to immediately call up a desired mode to the display 131 by the clinician for assistance in a medical procedure. In some embodiments, the display screen 131 is an LCD device.

Optionally, a stand-alone ultrasound probe 280 can be communicatively coupled with the console 130 by way of one of the ports 264. The ultrasound probe 280 may be employed in connection with ultrasound-based visualization of the target area as further described below.

In some embodiments, the console 130 may be a general computing device, such as a personal computer or a server, for example. The general computing device may be configured to communicatively couple with the temperature scanning device, and the optional ultrasound probe via a wired or wireless connection. In such embodiments, the logic 263 may be stored in memory of the general computing device and cause one or more processors of the general computing device to perform operations of the system 100.

As discussed above the temperature source 150 may take several forms. In FIG. 1A, the temperature source is illustrated an object grasped by the patient's hand. Such an object may be water bottle, an ice pack, a metal bar, or the like. As the hand comprises a large amount of capillary blood vessels, grasping the temperature source 150 may be effective in exchanging thermal energy with the blood flowing within the hand.

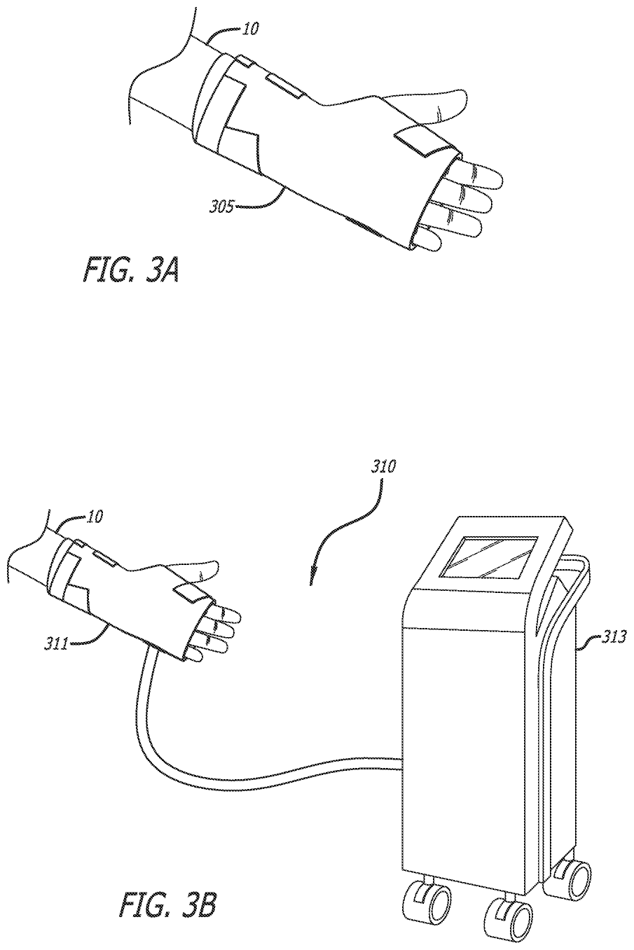

FIGS. 3A-3B illustrate additional embodiments of the temperate source 150. FIG. 3A illustrates a pad 305 wrapped around the hand thereby effecting the temperature of the palm and backside of the hand as well as the wrist. As such, the pad 305 may affect the temperature of the blood within the hand a greater rate than temperature source simply grasped by the hand. The pad 305 may be a cooling pad or a heating pad.

FIG. 3B illustrates a thermal contact pad 311 of a targeted temperature management system 310. The targeted temperature management system 310 includes a module 313 that circulates water having a defined temperature through the thermal contact pad 311 to affect the temperature of the thermal contact pad 311 and thereby, facilitate thermal energy exchange with the hand including the blood flowing through the hand. The targeted temperature management system 310 provides an advantage of setting the temperature of the temperature source 150 and adjusting the temperature as needed during the procedure.

Although the various embodiments of the temperature source 150 are shown and described as applied to the hand of the patient. Other embodiments may be configured to engage the patient 10 at other locations in accordance with various medical procedures. Similarly, the temperature source 150 embodiments are illustrated in accordance with affecting the temperature of venous blood flow. Other embodiments may be configured to affect the temperature of arterial blood flow.

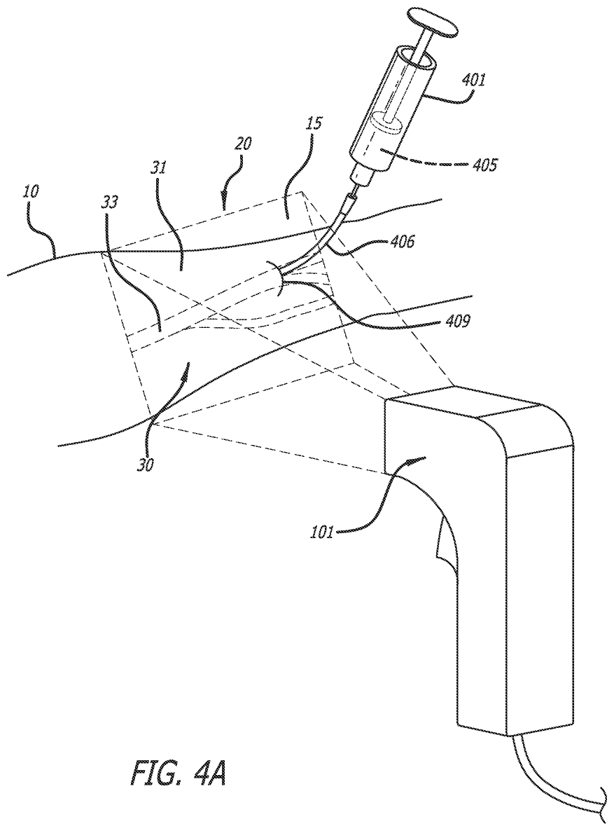

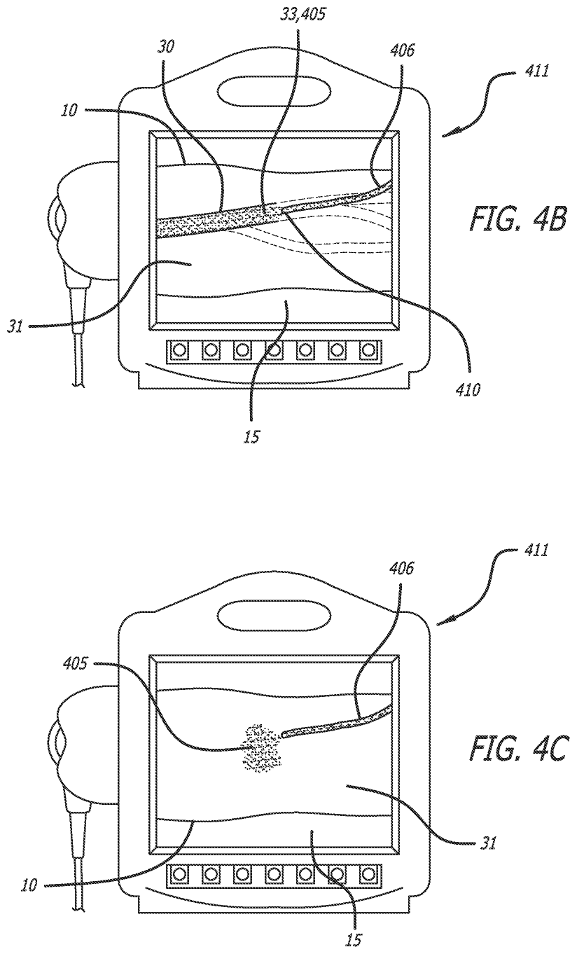

FIG. 4C illustrates a second implementation of the medical system 100. In this implementation, an infusate 405 is being delivered to the patient 10 via a syringe 401. The infusate 405 has a temperature that is lower than the temperature of the tissue 31. Infiltration (i.e., the delivery of an infusate into the patient outside of the blood vessel) is common error that occurs during cannulation which sometimes ay result in a hazard to the patient. As a mitigation during cannulation, the clinician may seek to verify that a distal tip of the catheter 406 is inserted into the blood vessel 30. A common practice is to draw back a syringe plunger (not shown) to draw fluid back into the syringe (not shown), at which point the clinician can verify that blood is drawn back into the syringe indicating that the distal tip is located within the blood vessel. During delivery of the infusate, the distal tip of a catheter may become dislodged from the blood vessel resulting in infiltration. The system 100 facilitates a method of verifying the lack of infiltration or detecting the presence infiltration while infusate is being delivered.

In the illustrated exemplary implementation, an infusate 405 is delivered to the patient 10 via a catheter 406. The infusate 405 has a temperature (e.g., room temperature) that is lower than the temperature of the tissue 31 and the blood 33. The catheter 406 passes through the skin at an insertion site 409.

FIG. 4B illustrates a thermal image 401 in accordance with a first instance where the distal tip 410 of the catheter 406 is located within the blood vessel 30. The infusate 405 flowing through the catheter 406 joins the blood 33 flowing within the blood vessel 30 at the distal tip 410. When the infusate 406 joins the blood 33, the lower temperature of the infusate 405 causes a lowering of the temperature of the blood 33 downstream of the distal tip 410. As shown, the infusate 405, due to its temperature difference in relation to the tissue 31, is visible in the thermal image 401. Similarly, the blood 33 downstream of the distal tip 410 is also visible in the thermal image 401 due to its temperature difference in relation to the body tissue 31. As such, the clinician may verify via the thermal image 401 that the infusate 405 is flowing into the blood vessel 30, i.e., infiltration is not occurring.

FIG. 4C illustrates a thermal image 411 in accordance with an alternative second instance where the distal tip 410 of the catheter 406 is dislodged from or otherwise not located within the blood vessel 30. The infusate 406 flowing through the catheter 406 flows into the tissue 31 causing an accumulation of infusate 406 adjacent the distal tip 410. The accumulation of the infusate 406 has a temperature that is lower than the temperature of the tissue 31. As shown, the accumulation of the infusate 405, due to its temperature difference in relation to the tissue 31, is visible in the thermal image 401. As such, the clinician may detect that infusate 405 is accumulating within the tissue 31 and not flowing into the blood vessel, i.e., that infiltration is occurring. By way of summary, the clinician can quickly and easily detect infiltration or verify the absence of infiltration, by observing a thermal image of the target area 20 obtained by the system 100.

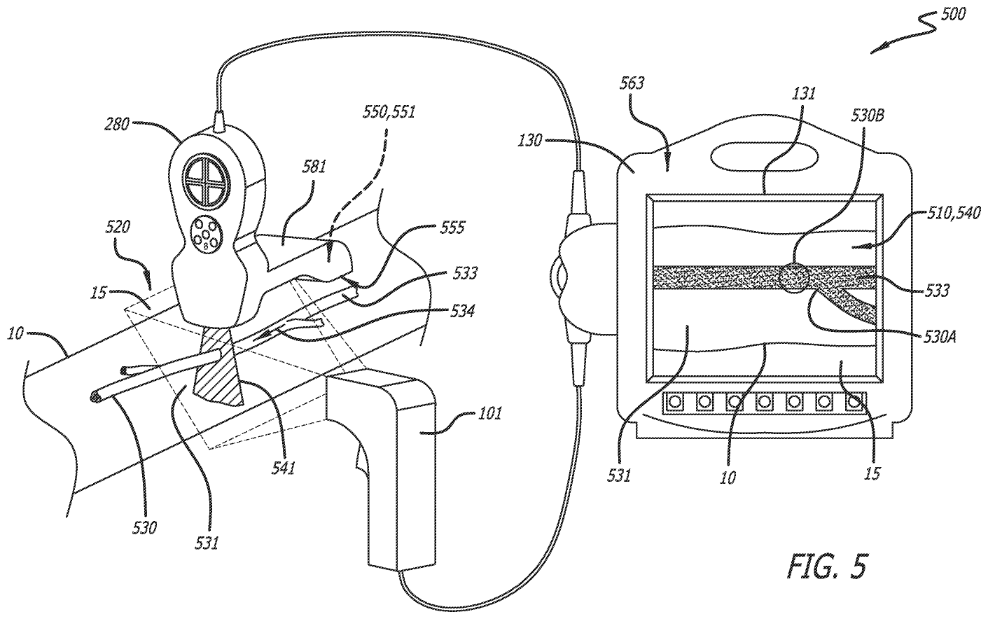

FIG. 5 illustrates a second embodiment of the medical system. The medical system can, in certain respects, resemble components of the medical system 100 described in connection with FIGS. 1A-4. It will be appreciated that all the illustrated embodiments may have analogous features. As such, relevant disclosure set forth above regarding similarly identified features thus may not be repeated hereafter. Moreover, specific features of the medical system 100 and related components shown in FIGS. 1A-4 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the medical system of FIG. 5. Any suitable combination of the features, and variations of the same, described with respect to the medical system 100 and components illustrated in FIGS. 1A-4 can be employed with the medical system and components of FIG. 5, and vice versa.

The medical system 500 generally includes the temperature scanning device 101 and the console 130 having logic 563 stored in memory of the console 130. The medical system 500 further includes the ultrasound probe 280 (see FIG. 2) coupled with the console 130. The ultrasound probe 280 is configured to obtain an ultrasound image of the target area 520 of the patient 10. FIG. 5 illustrates the system 500 obtaining a thermal image 510 of the target area 520 and further obtaining an ultrasound image 540 of the target area 520. The target area 520 includes a blood vessel 530 through which blood 533 is flowing in the direction indicated by the arrow 534.

In some embodiments, the ultrasound probe 280 includes a temperature source 550 integrated into the housing 581 of the ultrasound probe 280. The ultrasound probe 280 is configured such that the temperature source 550 is spaced laterally away from the ultrasound imaging plane 541. In use the ultrasound probe 280 may be placed in contact with the patient so that the imaging plane 541 and the temperature source 550 are aligned with the vasculature of the patient 10. In the illustrated implementation, the imaging plane 541 intersects the blood vessel 530 so that the ultrasound image 540 includes the blood vessel 530.

Similarly, the temperature source 550 is in contact with the patient 10 adjacent the blood vessel 530 so the temperature source 550 affects the temperature of the blood 530. In the illustrated instance, the temperature source 550 is also located upstream with respect to the blood flow direction so that blood 533 affected by the temperature source 550 flows through the target area 520 and more specifically the image plane 541.

The temperature source 550 may be configured to warm or cool the blood 533 or both. In some embodiments, the temperature source 550 may include a thermo-electric device (TED) 551 that defines a temperature of the bottom surface 555 of the housing 581. The TED 551 may be configured to define a temperature of the bottom surface 555 based on a DC voltage applied to the TED 551. Furthermore, the TED 551 may be configured to cool the blood 533 in accordance with a first polarity of the DC voltage and warm the blood in accordance with an opposite second polarity of the DC voltage.

According to exemplary use case, the temperature source 550 cools the blood 533 flowing within the blood vessel 530 to establish a temperature difference between the blood 533 and the surrounding tissue 531 within the target area 520. The temperature scanning device 101 detects the temperature difference and provides thermal imaging data to the console 130 accordingly where the logic 563 processes the thermal imaging data into a thermal image 510 to be portrayed on the display 131. At the same time, the ultrasound probe 280 provides ultrasound imaging data to the console where the logic 563 processes the ultrasound imaging data into an ultrasound image 540 to be portrayed on the display 131. The logic 563 may then superimpose the thermal image 510 atop the ultrasound image 540 (or vice versa) so that the clinician may view the blood flow image 530A as portrayed in the thermal damage 510 in combination with the blood vessel image 530B portrayed within the ultrasound image 540. By simultaneously observing the blood flow image 530A along with the blood vessel image 530B, the clinician may gain confidence that the blood vessel image 530B is consistent with a target blood vessel for a given medical procedure thereby avoiding a cannulation error, for example.

In some embodiments, the logic 563 may perform a spatial positioning assess of the blood flow image 530A with respect to the blood vessel image 530B thereby determine confidence that the blood flow image 530A is in assignment with the blood vessel image 530B.

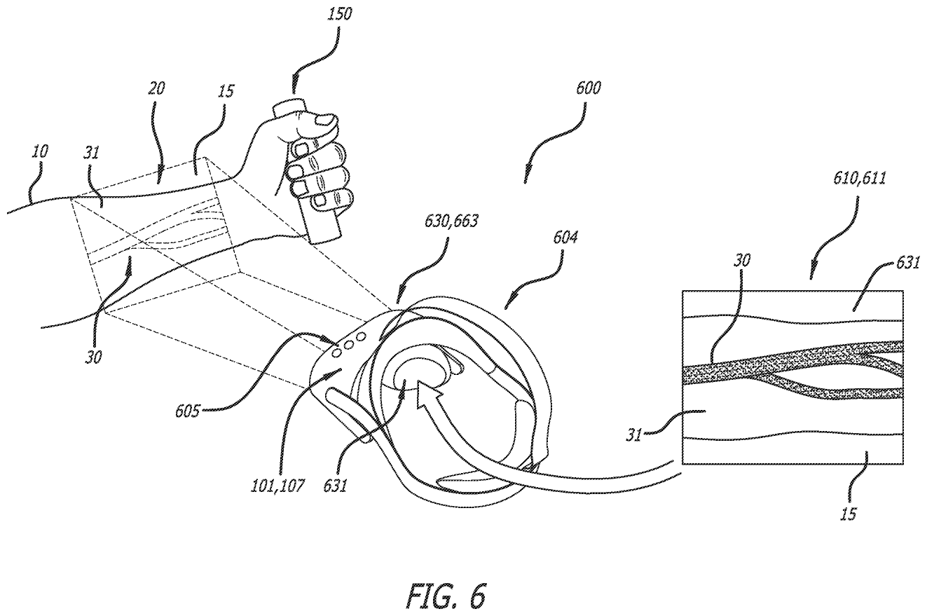

FIG. 6 illustrates a third embodiment of the medical system. The medical system 600 can, in certain respects, resemble components of the medical systems 100, 500 described in connection with FIGS. 1A-5. It will be appreciated that all the illustrated embodiments may have analogous features. As such, relevant disclosure set forth above regarding similarly identified features thus may not be repeated hereafter. Moreover, specific features of the medical systems 100, 500 and related components shown in FIGS. 1A-5 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the medical system of FIG. 6. Any suitable combination of the features, and variations of the same, described with respect to the medical systems 100, 500 and components illustrated in FIGS. 1A-5 can be employed with the medical system and components of FIG. 6, and vice versa.

The medical system 600 generally includes the temperature scanning device 101, a console 630, and a display 631 enclosed with a headset housing 604. The system 600 may include one or more input devices 605 (e.g., control buttons) located on the hosing 604 for defining operating modes of the medical system 600. In some embodiments, the input devices 605 may include a button for obtaining snapshots of the augmented reality view.

The display 631 may be a single display within the housing a pair of displays (e.g., one for each eye). The logic 663 may cause the thermal image 610 to be portrayed on the display 631. As such, the clinician may identify and locate the blood vessels 30 within the target area 20 of the patient 10 as described above in relation to the systems 100, 500.

In a similar fashion to the system 100, although not required, the system 600 may include the camera 107 according to some embodiments. The camera 107 may be included within the housing 604. The camera 107 may is configured to obtain a camera image 611 of the target area 20. In some embodiments, the camera image 611 may be portrayed in combination with the thermal image 610, such as superimposed atop the thermal image 610 or vice versa. As such, the system 600 may operate similar to a virtual reality device.

According to alternative embodiment, the system 600 may operate as an augmented reality device. In such an embodiment, the system 600 includes lenses (e.g., glasses not shown) included in the housing 604 through which the clinician may be directly view the target area 20. The display 631 projects the thermal image 610 onto the lenses (or otherwise makes the thermal image 610 visible) so that the clinician may view the thermal image 610 in combination with the direct view of the target area. As such, the clinician may identify and locate the blood vessels 30 within the target area 20 of the patient 10.

Methods of the foregoing medical systems include methods implemented in the medical systems. For example, a method of the medical system (e.g., systems 100, 500, or 600) includes a non-transitory CRM (e.g., EEPROM) having the logic stored thereon that causes the medical system to perform a set of operations for thermal imaging when the logic is executed by the processors of the console.

The logic may obtain temperature data pertaining to subcutaneous blood vessels within a target area when the temperature of the blood within the blood vessels is altered via a temperature source. The logic may process the temperature data to determine the location of the blood vessels. The logic may further provide an indication pertaining to the location of the blood vessels to the clinician. The logic may then generate an image or mapping of the blood vessels for portrayal on the display so that the clinician may visualize the blood vessels within the target area.

In some embodiments, the logic may obtain a camera image of the target area of the patient along with obtaining the thermal image of subcutaneous blood vessels. The logic may then portray the thermal image in combination with camera image on the display of the system.

In some embodiments, the logic may obtain an ultrasound image of the target area via an ultrasound probe. The logic may then portray the thermal image in combination with the ultrasound image on the display.

Use of the system (e.g., systems 100, 500, or 600) also includes operations performed by the clinician to facilitate the locating a desired subcutaneous blood vessel in preparation for performing an intravascular medical procedure.

In general, the clinician may determine a vascular condition of a patient by causing a temperature difference within a subcutaneous target area of the patient, obtaining a thermal image of a skin surface adjacent the subcutaneous target area via the system, and then by observation of the thermal image, the clinician may determine the vascular condition based on the thermal image.

The clinician may apply a temperature source to the patient at a location that is upstream of an intended insertion site for a cannula to be inserted into a predefined blood vessel. In so doing, the clinician may lower the temperature of the blood flowing within the predefined blood vessel. The lowered temperature of the blood migrates to the skin surface causing variations in the temperature of the skin surface where they are detected by the temperature scanning device so that the location of the blood vessels are made visible.

Based on the location of the temperature source on the patient, the clinician may identify the blood vessel as a vein or an artery based on the position of the temperature source. In other words, as the temperature source affects only the blood temperature upstream the target area, the clinician may determine that the any blood vessels that are portrayed in the thermal image have a blood flow direction from the temperature source toward the target area.

As the thermal image portrays the blood vessel and the outer limits of the patient within the target area (i.e., the transition in temperature between the skin surface the local environment), the clinician may determine by observation of the thermal image, the location of the blood vessel within the target area.

Methods may also include identifying a vein directly by placing the temperature source upstream of the target area with respect to venous flow generally. Similarly, the clinician may directly identify an artery by placing the temperature source upstream of the target area with respect to arterial flow generally.

In some instances, the blood may exit the blood vessel via an opening in the vessel wall causing. By placing the temperature source upstream with respect to the blood flow within a blood vessel of concern, the clinician may observe any occurring extravasation emanating from the blood vessel within the thermal image and thereby determine any extravasation.

An infusate may generally be delivered at room temperature (i.e., lower than blood or body tissue temperature). As such, the clinician may view the infusate within the blood vessel during delivery. Similarly, in an instance of infiltration of the infusate, the clinician may observe the infiltration within the thermal image.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A medical system, comprising:
a temperature scanning device configured to provide temperature data based on temperature variations of a skin surface extending across a subcutaneous target area of a patient body; and
a console configured to communicate with the temperature scanning device, the console including one or more processors and a non-transitory computer-readable medium having stored thereon logic that, when executed by the one or more processors, causes operations including:
obtaining temperature data across the target area, the target area including a subcutaneous blood vessel, wherein a cause of the temperature variations across the target area include a temperature difference of a blood temperature within the blood vessel with respect to a body tissue temperature adjacent the blood vessel.

2. The medical system of claim 1, wherein the operations further include providing an indication to the clinician based on the temperature data.

3. The medical system of claim 2, wherein the operations further include:
converting the temperature data into thermal image data, and
providing a thermal image to the clinician based on the thermal image data.

4. The medical system of claim 3, wherein the thermal image includes a mapping of the blood vessels within the target area.

5. The medical system of claim 3, wherein providing the thermal image includes providing a live thermal image.

6. The medical system of claim 3, wherein providing the thermal image includes providing a thermal image snapshot.

7. The medical system of claim 3, wherein providing the thermal image includes portraying the thermal image on a display of the system.

8. The medical system of claim 1, further including a temperature source configured to alter the blood temperature within the blood vessel.

9. The medical system of claim 8, wherein the blood temperature is lower than the temperature of the body tissue.

10. The medical system of claim 8, wherein:
the blood temperature is altered by placing the temperature source in contact with a target contact area of the patient, and
the target contact area is located upstream with respect to a blood flow within the blood vessel.

11. The medical system of claim 8, wherein the temperature source is one of a hot pack or a cold pack.

12. The medical system of claim 8, wherein the temperature source is a thermal contact pad of a targeted temperature management system.

13. The medical system of claim 8, wherein the temperature source is an infusate delivered to the blood vessel.

14. The medical system of claim 1, wherein the cause of the temperature variations includes a temperature gradient extending along the blood vessel.

15. The medical system of claim 1, wherein the cause of the temperature variations include a difference between a temperature of a blood volume outside of the blood vessel and the body tissue temperature adjacent the blood vessel.

16. The medical system of claim 1, wherein the cause of the temperature variations include an infusate temperature within the blood vessel with respect to the body tissue temperature adjacent the blood vessel.

17. The medical system of claim 1, wherein the console is included within a housing of the temperature scanning device.

18. The medical system of claim 1, wherein a display of the system is included within the housing of the temperature scanning device.

19. The medical system of claim 1, further comprising a camera for providing a camera image of the target area.

20. The medical system of claim 19, wherein the camera is included within the housing of the temperature scanning device.

21. The medical system of claim 19, wherein the operations further include:
obtaining a camera image of the target area; and
providing the thermal image superimposed atop the camera image.

22. The medical system of claim 1, further comprising an ultrasound probe coupled with the console, the ultrasound probe configured to obtain an ultrasound image of the target area.

23. The medical system of claim 22, wherein the operations further include:
obtaining an ultrasound image of the target area; and
providing the thermal image superimposed atop the ultrasound image.

24. The medical system of claim 22, wherein the temperature source is integrated into the ultrasound probe.

25. The medical system of claim 1, wherein the temperature scanning device is a hand-held device.

26. The medical system of claim 1, further comprising an augmented reality device, wherein:
the temperature scanning device is coupled with the augmented reality device; and
an augmented reality image of the target area includes a mapping of the blood vessels overlayed onto a view of the target area.

27-35. (canceled)
